(19) United States
(12) Patent Application Publication
(10) Pub. No.: US 2016/0074070 A1
(43) Pub. Date: Mar. 17, 2016

(54) MENISCAL REPOSITIONING DEVICE

(71) Applicant: Empire Technology Development LLC, Wilmington, DE (US)

(72) Inventor: Nicky Bertollo, Wollstonecraft (AU)

(21) Appl. No.: 14/485,518

(22) Filed: Sep. 12, 2014

(51) Int. Cl. A61B 17/56

(52) U.S. Cl. CPC A61B 17/562; A61B 2017/564

(57) ABSTRACT

Technologies are generally provided for a meniscal repositioning device to achieve at least a partial unloading of an ipsilateral tibiofemoral articulation. The device may include at least a shortening component and a fixation component. The shortening component may be configured to reduce a functional length of a meniscal root of a meniscus through an introduction of a targeted and strategic deviation in the device associated with an accommodation of the meniscal root. The deviation may cause a path of at least one portion of the meniscus to be altered, further causing the meniscus to be drawn inward towards a midline of a knee to induce a distraction force in a medial or lateral compartment of the knee between menisci. The fixation component may affix the shortening component to a portion of a tibia adjacent to the meniscal root or an anterior and/or posterior portion of the meniscal root.

### BACKGROUND

Unless otherwise indicated herein, the materials described in this section are not prior art to the claims in this application and are not admitted to be prior art by inclusion in this section.

Menisci of a knee include a lateral meniscus (LM) and a medial meniscus (MM), which are crescent-shaped fibrocartilaginous structures that partially divide joint space of the knee. The menisci are flattened at a center of the knee joint, fused with a synovial membrane laterally, and may translate over a tibial surface, such as a tibial plateau. The menisci of the knee provide a crucial biomechanical role, the most important of which may include load transmission and shock absorption. For example, when a person is upright, as much as 55% of the downward pressure exerted by a femoral condyle on an articular surface of the tibial plateau may be dissipated by a stretching of circumferential collagen fiber bundles that comprise a central portion of the menisci. The menisci of the knee may further reduce wear by converting an axial load to a radially directed force or hoop stress.

A severely arthritic knee may be characterized by bone on bone contact in a medial or lateral compartment of the knee, due to deterioration of femoral and tibial cartilage, having negative implications for pain and mobility. A tissue sparing device, that is temporary in nature, may be desired to stave off the progression of osteoarthritis, decrease pain, restore functionality, and therefore postpone end-stage osteoarthritis treatment with total knee replacement (TKR) surgery.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

According to some examples, a meniscal repositioning device may be described. An example meniscal repositioning device may include a shortening component comprising a semi-circular channel on an interior surface of the shortening component, the semi-circular channel configured to accommodate a passage of a meniscal root. The shortening component may be configured to alter a path of at least one portion of a meniscus in order to reduce a functional length of the meniscal root such that at least a partial unloading of the ipsilateral tibiofemoral articulation is achieved. The example meniscal repositioning device may also include a fixation component configured to affix the shortening component to a portion of a tibia adjacent to the meniscal root, an anterior portion of the meniscal root, and/or a posterior portion of the meniscal root.

According to other examples, a meniscal repositioning device to achieve unloading of an ipsilateral tibiofemoral articulation may be described. An example meniscal repositioning device may include a shortening component comprising a curved channel on an exterior surface of the shortening component, the curved channel configured to accommodate a passage of a meniscal root. The shortening component may be configured to alter a path of at least one portion of a meniscus in order to reduce a functional length of a meniscal root such that at least a partial unloading of the ipsilateral tibiofemoral articulation is achieved. The example meniscal repositioning device may also include a fixation component configured to affix the shortening component to a portion of a tibia adjacent to the meniscal root.

According to further examples, a method to reposition a meniscus to achieve partial unloading of an ipsilateral tibiofemoral articulation may be provided. An example method may include inserting a shortening component adjacent to a meniscal root, the shortening component configured to alter a path of at least one portion of the meniscus in order to reduce a functional length of the meniscal root such that at least a partial unloading of the ipsilateral tibiofemoral articulation is achieved. The shortening component may include a semi-circular channel on an interior surface or a curved channel on an exterior surface of the shortening component configured to accommodate a passage of the meniscal root. The example method may also include affixing the shortening component to a portion of a tibia adjacent to the meniscal root employing a fixation component.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIGS. 1A and 1B illustrate an example superior view of menisci and associated meniscal roots and a meniscal repositioning device in situ;

FIG. 2 illustrates an example meniscal repositioning device;

FIG. 3 illustrates an altered path of a meniscal root passing through an example meniscal repositioning device;

FIG. 4 illustrates an example configuration of a meniscal repositioning device;

FIGS. 5A and 5B illustrate an example configuration of a meniscal repositioning device configured to bridge anterior or posterior meniscal roots of menisci;

FIGS. 6A and 6B illustrate an example configuration of a meniscal repositioning device as a mechanical cam;

FIG. 7 illustrates an altered path of a meniscal root employing a mechanical cam meniscal repositioning device;

FIG. 8 illustrates an example configuration of a meniscal repositioning device as a condensed mechanical cam;

FIGS. 9A and 9B illustrate another example configuration of a meniscal repositioning device;

FIG. 10 illustrates an example configuration of a meniscal repositioning device including sutures for fixation;

FIG. 11 illustrates an example configuration of a meniscal repositioning device inserted in a bisected meniscal root;

FIG. 12 illustrates an example configuration of a meniscal repositioning device including support members inserted in a bisected meniscal root;

FIG. 13 illustrates an example configuration of a meniscal repositioning device including movable prongs; and

FIG. 14 illustrates an altered path of a meniscal root employing a staple as a meniscal repositioning device;

all arranged in accordance with at least some embodiments as described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, apparatus, systems, and/or devices related to providing a meniscal repositioning device to achieve at least a partial unloading of an ipsilateral tibiofemoral articulation.

Briefly stated, technologies are generally provided for a meniscal repositioning device to achieve at least a partial unloading of an ipsilateral tibiofemoral articulation. The device may include at least a shortening component and a fixation component. The shortening component may be configured to reduce a functional length of a meniscal root of a meniscus through an introduction of a targeted and strategic deviation in the device associated with an accommodation of the meniscal root. The deviation may cause a path of at least one portion of the meniscus to be altered, further causing the meniscus to be drawn inward towards a midline of a knee to induce a distraction force in a medial or lateral compartment of the knee between menisci. The fixation component may affix the shortening component to a portion of a tibia adjacent to the meniscal root or an anterior and/or posterior portion of the meniscal root.

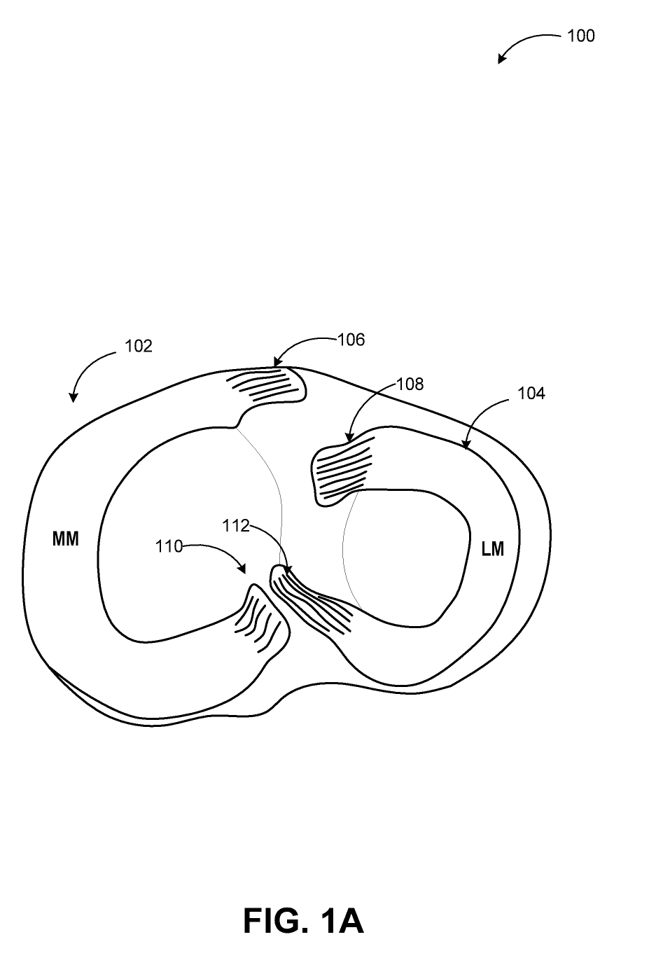

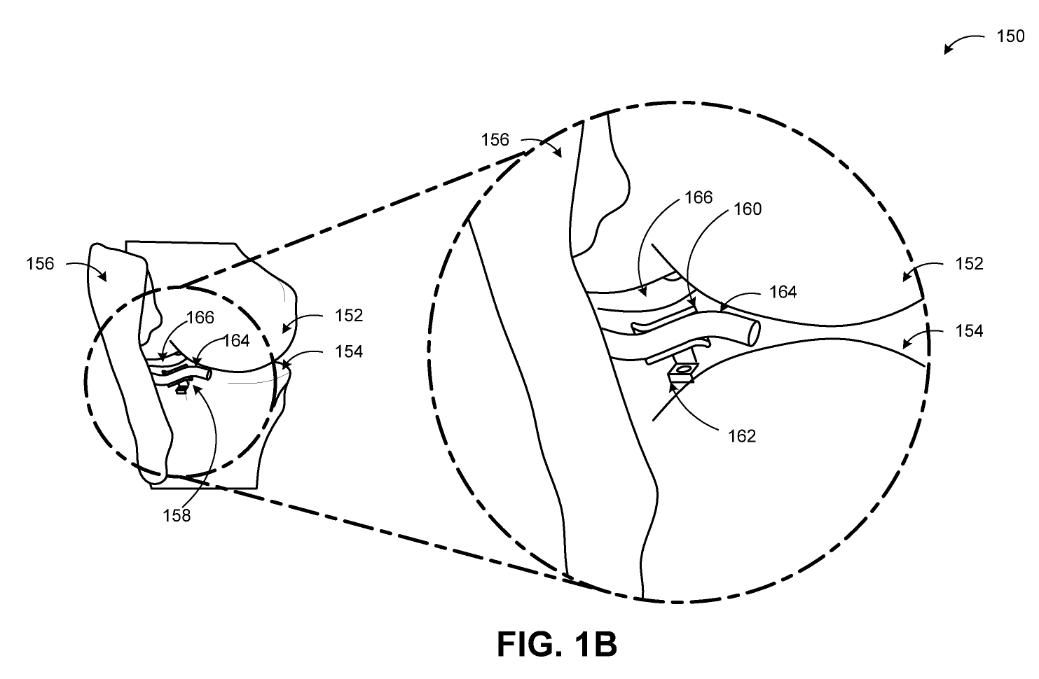

FIGS. 1A and 1B illustrate an example superior view of menisci and associated meniscal roots and a meniscal repositioning device in situ, arranged in accordance with at least some embodiments as described herein.

As shown in FIG. 1A, diagram 100, menisci of a knee may include a medial meniscus (MM) 102 and a lateral meniscus (MM) 104. The MM 102 may include an anterior meniscal root 106 and a posterior meniscal root 110. The LM 104 may similarly include an anterior meniscal root 108 and a posterior meniscal root 112.

The menisci, MM 102 and LM 104, may be superior to a tibial plateau of a tibia, and may be tethered to a center of the tibial plateau through fibers originating from the anterior meniscal roots 106, 108 and the posterior meniscal roots 110, 112. The menisci may be inferior to lateral and medial condyles of a femur, where the anterior meniscal root 106 of the MM 102 may insert broadly on an anterior intercondylar crest of the tibia. The meniscal roots 106, 108, 110, 112 may further tether the MM 102 and the LM 104 together, while still allowing limited translation in mediolateral and anteroposterior dimensions.

A severely arthritic knee may be characterized by bone on bone contact between the femur and tibia in a medial or lateral compartment of the knee, due to deterioration of femoral and tibial cartilage, having negative implications for pain and mobility. A temporary tissue sparing device, such as a meniscal repositioning device to be discussed in conjunction with FIG. 1B, may be employed to stave off the progression of osteoarthritis, decrease pain, restore functionality, and therefore postpone end-stage osteoarthritis treatment with total knee replacement (TKR) surgery.

As shown in FIG. 1B, diagram 150, an example meniscal repositioning device may include meniscal repositioning device 158. As illustrated, the meniscal repositioning device 158 may be positioned in situ within a medial compartment of a knee such that the meniscal repositioning device 158 is inferior to a medial condyle of a femur 152, superior to a tibial plateau of a tibia 154, posterior to a patella 156, and medial to a LM 166, such that passage of a meniscal root 164 of an MM may be accommodated by the meniscal repositioning device 158. However, in other embodiments, the meniscal repositioning device 158 may be positioned in situ within a lateral compartment of a knee such that the meniscal repositioning device 158 is inferior to a lateral condyle of a femur, superior to a tibial plateau of a tibia, posterior to a patella, and medial to the LM 166. The meniscal repositioning device 158 may include at least a shortening component 160 and a fixation component 162. The meniscal repositioning device 158 may be composed of stainless steel, nickel titanium alloy, titanium, cobalt-chrome, a polymer, a ceramic material, and a composite material, for example. The meniscal repositioning device 158 may be symmetrical such that the meniscal repositioning device 158 affects fibers of the meniscal root 164 to a same extent, producing an even strain distribution in the structure.

The shortening component 160 may include a semi-circular channel on an interior surface of the shortening component 160 configured to accommodate the passage of the meniscal root 164 of the meniscus, where the meniscus may be the MM or the LM 166. In other embodiments, the shortening component may include one or more semi-circular channels on an interior surface and/or one or more curved channels on an exterior surface of the shortening component. The meniscal root 164 may be an anterior meniscal root or a posterior meniscal root. In some examples, the shortening component 160 may be configured to accommodate a passage of more than one meniscal root 164, and in such examples the meniscal roots may be anterior and/or posterior meniscal roots. The shortening component 160 may be further configured to alter a path of at least one portion of the meniscus, where the at least one portion may include the meniscal root 164, in order to reduce a functional length of the meniscal root 164. An axial force associated with the fixation component 162 screwing and/or seating the meniscal repositioning device 158 into position may induce the reduction of the functional length of the meniscal root, where an extent of the length reduction may be adjusted by changing a length and/or angulation of the semi-circular channel of the shortening component 160 relative to a horizontal plane.

By reducing the functional length of the meniscal root 164, the at least one portion of the meniscus may be drawn inward towards a midline of a knee to induce a distraction force in either the medial compartment or lateral compartment of the knee between the MM and the LM 166 enabling the partial unloading of the ipsilateral tibiofemoral articulation. In some examples, the shortening component 160 may include one or more prongs and/or protrusions to enable interaction between the meniscal repositioning device 158 and a transverse genual ligament that links the anterior portions of the medial and lateral menisci.

In further embodiments, the meniscal repositioning device 158 may include two shortening components, one lateral to the MM and one medial to the LM, such that paths of at least two or more portions of the meniscus are altered to a substantially same degree in order to reduce a strain across the menisci.

The fixation component 162 may be configured to affix the shortening component 160 to a portion of the tibia 154, such as the tibial plateau, adjacent to the meniscal root 164, as illustrated. In other examples, the shortening component 160 may deviate in structure such that the fixation component 162 may be configured to affix the shortening component 160 to an anterior portion of the meniscal root 164 and/or a posterior portion of the meniscal root 164. As further illustrated, the fixation component 162 may include an anchor component attached to an inferior surface of the semi-circular channel of the shortening component 160, the anchor component configured to enable fixation of the shortening component 160 to the tibial plateau of the tibia 154 through a cortical or cancellous bone screw inserted through a corresponding hole in the anchor component. In some examples, the fixation component 162 may include a male Morse taper at a proximal end configured to permit relative rotation of the shortening component 160 to obtain a desired path alteration of the at least one portion of the meniscus, and to permit a locking mechanism to fix the shortening component 160 in a particular position. In other examples, the fixation component may include a series of sutures accommodated by a plurality of recesses in the shortening component 160 configured to stabilize the shortening component 160 in a desired position by attaching the shortening component 160 to an anchor component comprising a screw post, a bar, and/or a suture anchor implanted into the tibial plateau of the tibia 154 near the LM 166.

The meniscal repositioning device 158 may effectively manipulate biological structures, such as the LM 166 and/or the MM, already present in the knee to achieve the partial unloading of the ipsilateral tibiofemoral articulation in a medial and/or lateral compartment of the knee. By decreasing the functional length of the anterior and/or posterior meniscal root of the LM 166 and/or the MM, the LM 166 and/or the MM may be drawn to the midline of the knee, thereby wedging apart the medial and/or lateral compartment of the knee and providing at least the partial unloading of the ipsilateral tibiofemoral articulation through an increase in the percentage load borne by the LM 166 and/or the MM. Such an approach manipulating biological structures may be preferred to TKR surgery, as the menisci are removed during TKR, which alleviates concerns regarding increased load and potentially accelerated degradation of the meniscus which may be caused by the induced shift towards the midline of the knee.

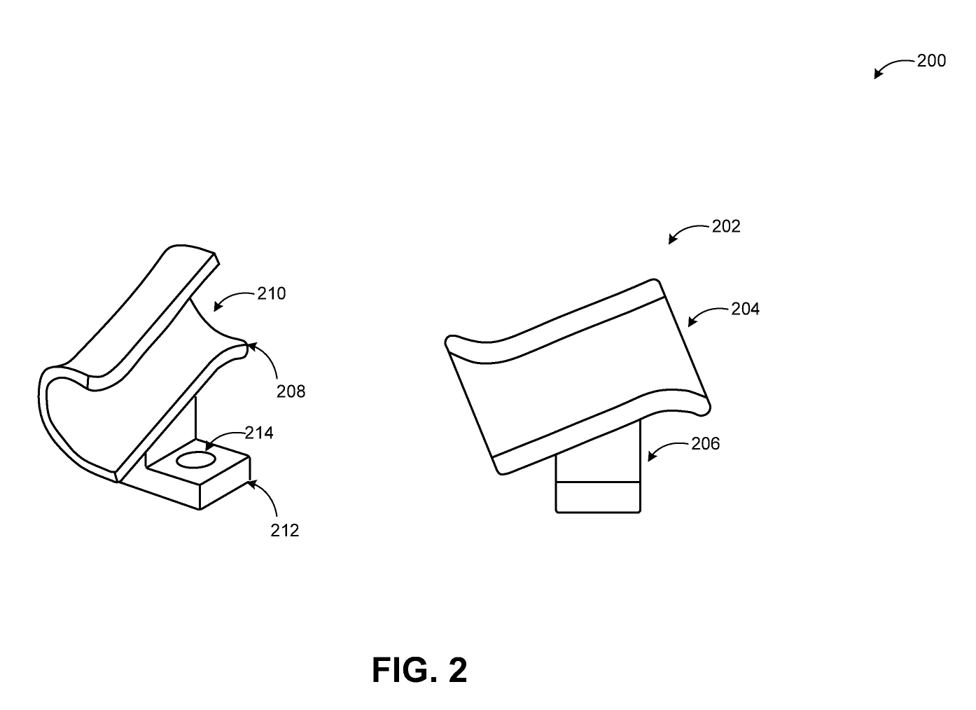

FIG. 2 illustrates an example meniscal repositioning device, arranged in accordance with at least some embodiments as described herein.

As shown in a diagram 200, a meniscal repositioning device 202 may include at least a shortening component 204 and a fixation component 206. The shortening component 204 may include a semi-circular channel 210 on an interior surface of the shortening component 204 configured to accommodate a passage of a meniscal root of a meniscus, where the meniscus may be a LM or a MM and the meniscal root may be an anterior meniscal root or a posterior meniscal root. The shortening component 204 may be substantially elongated such that the meniscal root is enabled to translate in a substantially perpendicular direction with respect to the semi-circular channel 210 helping to facilitate relative medial-lateral and anteroposterior translation motion between the LM and/or MM and a tibial plateau which normally occurs with knee function. Furthermore, a first end and a second end of the semi-circular channel 210 may be in a substantially same horizontal plane to ensure no extraneous forces or over-constraints may be introduced, other than the intended effect of reducing a functional length of the meniscal root. The semi-circular channel 210 may include a series of smooth contours 208 to facilitate altering the path of at least one portion of the meniscus, which may include the meniscal root, as the meniscal root passes through the semi-circular channel 210 in order to reduce the functional length of the meniscal root. An axial force associated with the fixation component 206 screwing and/or seating the meniscal repositioning device 202 into position may induce the reduction of the functional length of the meniscal root, where an extent of the length reduction may be adjusted by changing a length and/or angulation of the semi-circular channel 210 of the shortening component 204 relative to the horizontal plane. By reducing the functional length of the meniscal root, the at least one portion of the meniscus may be drawn inward towards a midline of a knee to induce a distraction force in one of a medial compartment and a lateral compartment of the knee between the LM and the MM enabling the partial unloading of the ipsilateral tibiofemoral articulation. The induced distraction force may aid in preserving tissues while decreasing pain for a symptomatic patient.

The fixation component 206 may be configured to affix the shortening component 204 to a portion of a tibia adjacent to the meniscal root. In other embodiments, a structural deviation of the shortening component 204 may enable the fixation component 206 to be configured to affix the shortening component 204 to an anterior portion of the meniscal root 164 and/or a posterior portion of the meniscal root. As illustrated, the fixation component 206 may include an anchor component 212 attached to an inferior surface of the semi-circular channel 210 of the shortening component 204. The anchor component 212 may be configured to enable fixation of the shortening component 204 to the cortical bone of a tibial plateau of the tibia through a screw inserted through a corresponding recess 214 in the anchor component 212. The screw may be a cortical or cancellous bone screw, for example. To improve seating of the meniscal repositioning device 202 and fixation to the tibial plateau, an arthroscopic shaver or reamer may be introduced into the knee to bur the cortical bone of the tibial plateau in preparation for receiving the meniscal repositioning device 202. In some examples, an inferior surface of the anchor component 212 in contact with the tibial plateau may include a series of prongs to facilitate fixation to the adjacent tibia and/or an osteoconductive surface to promote bone growth to facilitate fixation with the adjacent tibia.

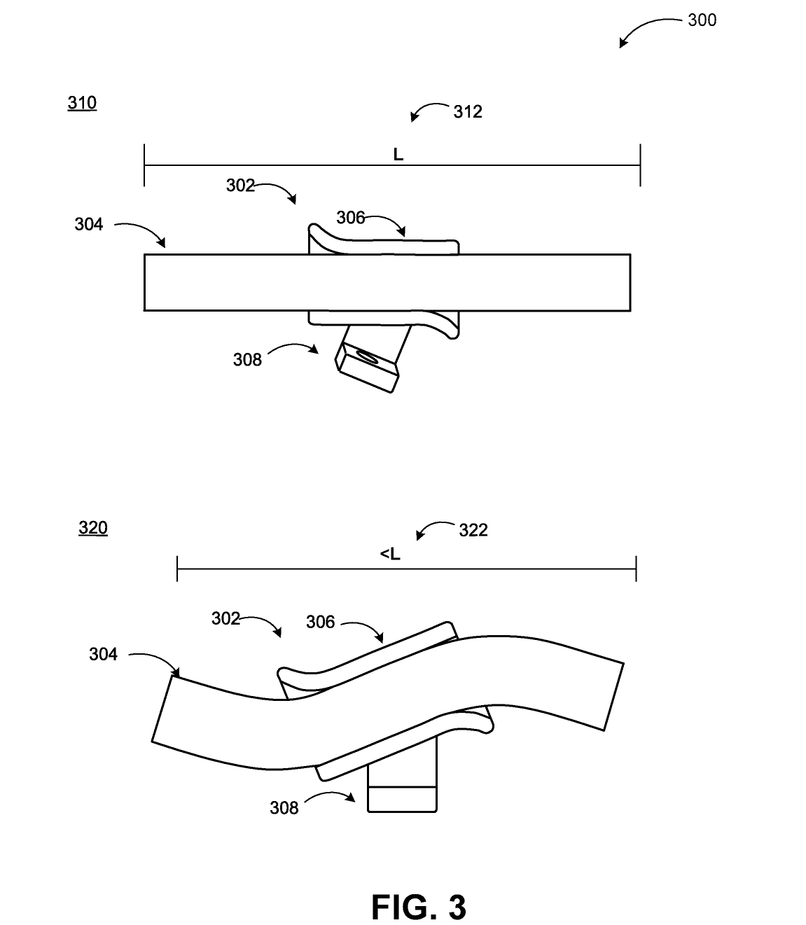

FIG. 3 illustrates an altered path of a meniscal root passing through an example meniscal repositioning device, arranged in accordance with at least some embodiments as described herein.

As shown in a diagram 300, a meniscal repositioning device 302 may include at least a shortening component 306 and a fixation component 308. The shortening component 306 may include a semi-circular channel on an interior surface of the shortening component 306 configured to accommodate a passage of a meniscal root 304 of a meniscus, where the meniscus may be a LM or a MM, and the meniscal root may be an anterior meniscal root or a posterior meniscal root.

In a first configuration 310, the shortening component 306 may be substantially elongated such that the meniscal root 304 is enabled to translate in a substantially perpendicular direction with respect to the semi-circular channel of the shortening component 306 helping to facilitate relative medial-lateral and anteroposterior translation motion between the LM and/or MM and a tibial plateau which occurs with normal knee function. Furthermore, a first end and a second end of the semi-circular channel of the shortening component 306 may be in a substantially same horizontal plane to ensure no extraneous forces or over-constraints may be introduced, other than the intended effect of reducing a functional length of the meniscal root 304. The functional length, L, of the meniscal root may be illustrated by distance 312. The fixation component 308 may be configured to affix the shortening component 306 to a portion of a tibia adjacent to the meniscal root 304. As illustrated, the fixation component 308 may include an anchor component attached to an inferior surface of the semi-circular channel of the shortening component 306. The anchor component may be configured to enable fixation of the shortening component 306 to a cortical bone of a tibial plateau of the tibia through a screw inserted through a corresponding recess in the anchor component.

In a second configuration 320, an axial force associated with screwing and seating the meniscal repositioning device 302 into position may induce the reduction of the functional length of the meniscal root 304, <L, as illustrated by distance 322 such that at least a partial unloading of the ipsilateral tibiofemoral articulation is achieved. By reducing the functional length of the meniscal root 304, at least one portion of the meniscus may be drawn inward towards a midline of a knee to induce a distraction force in one of a medial compartment and a lateral compartment of the knee between the LM and the MM enabling the partial unloading of the ipsilateral tibiofemoral articulation.

The extent of the length reduction may be adjusted by changing a length and/or angulation of the semi-circular channel of the shortening component 306 relative to the horizontal plane, as illustrated. A range of positional changes may be provided by a series of devices in which a length of the semi-circular channel of the shortening component 306 and/or angle of the long axis of the semi-circular channel relative to the horizontal plane is modified. Accordingly, an amount of intended positional change, and therefore unloading of the ipsilateral tibiofemoral articulation, may be tightly controlled and tailored based on individual patient anatomy and an integrity and/or state of the menisci. For example, an aggressive device with a substantially elongated and highly angulated semi-circular channel of the shortening component 306 relative to the horizontal plane may be indicated for a patient with healthy menisci, which may be determined from preoperative Magnetic Resonance Imaging (MRI) and/or Computerized Tomography (CT) scans. The semi-circular channel of the shortening component 306 may include a series of smooth contours to facilitate altering the path of the meniscal root 304 as the meniscal root passes through the semi-circular channel in order to reduce the functional length of the meniscal root.

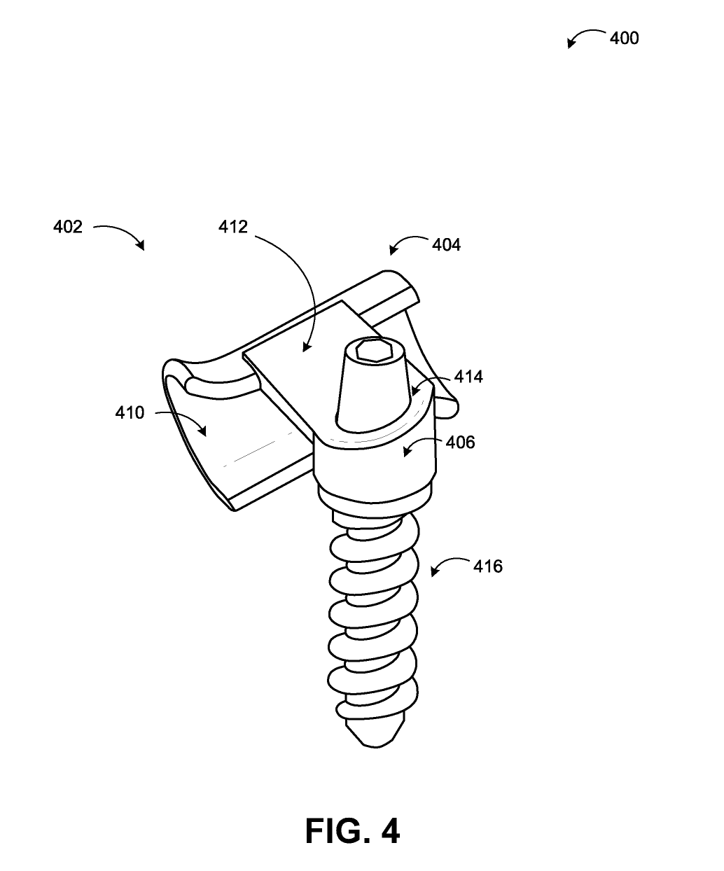

FIG. 4 illustrates an example configuration of a meniscal repositioning device, arranged in accordance with at least some embodiments as described herein.

As shown in a diagram 400, a meniscal repositioning device 402 may include at least a shortening component 404 and a fixation component 406. The shortening component 404 may include a semi-circular channel 410 on an interior surface of the shortening component 404 configured to accommodate a passage of a meniscal root of a meniscus, where the meniscus may be a LM or a MM and the meniscal root may be an anterior meniscal root or a posterior meniscal root. The semi-circular channel 410 may include a series of smooth contours to facilitate altering the path of at least a portion of the meniscus, which may include the meniscal root, as the meniscal root passes through the semi-circular channel 410 in order to reduce the functional length of the meniscal root. An axial force associated with the fixation component 406 screwing and/or seating the meniscal repositioning device 402 into position may induce the reduction of the functional length of the meniscal root, where an extent of the length reduction may be adjusted by changing a length and/or angulation of the semi-circular channel 410 of the shortening component 404 relative to a horizontal plane. By reducing the functional length of the meniscal root, the at least one portion of the meniscus may be drawn inward towards a midline of a knee to induce a distraction force in one of a medial compartment and a lateral compartment of the knee between the LM and the MM enabling the partial unloading of the ipsilateral tibiofemoral articulation.

The fixation component 406 may be configured to affix the shortening component 404 to a portion of a tibia adjacent to the meniscal root. As illustrated, the fixation component 406 may include an anchor component 412 attached to a superior surface of the semi-circular channel 410 of the shortening component 404. The anchor component 412 may be configured to enable fixation of the shortening component 204 to a cortical bone of a tibial plateau of the tibia through a screw 416 inserted through a corresponding recess 414 in the anchor component 412. The screw 416 may be a cortical or cancellous bone screw, for example. In some examples, an inferior surface of the anchor component 412 in contact with the tibial plateau may include a series of prongs to facilitate fixation to the adjacent tibia and/or an osteoconductive surface to promote bone growth to facilitate fixation with the adjacent tibia.

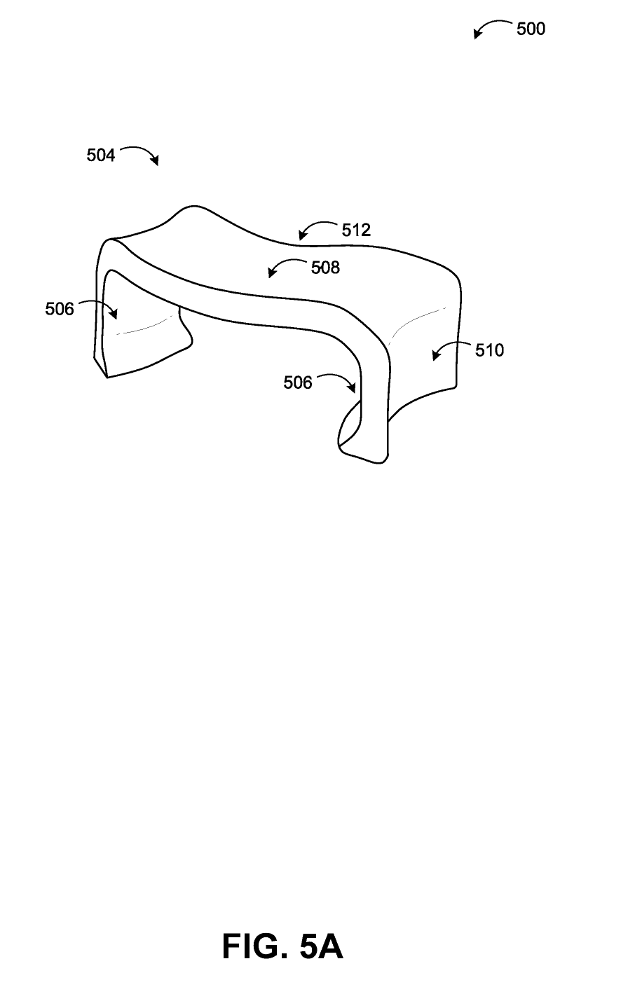

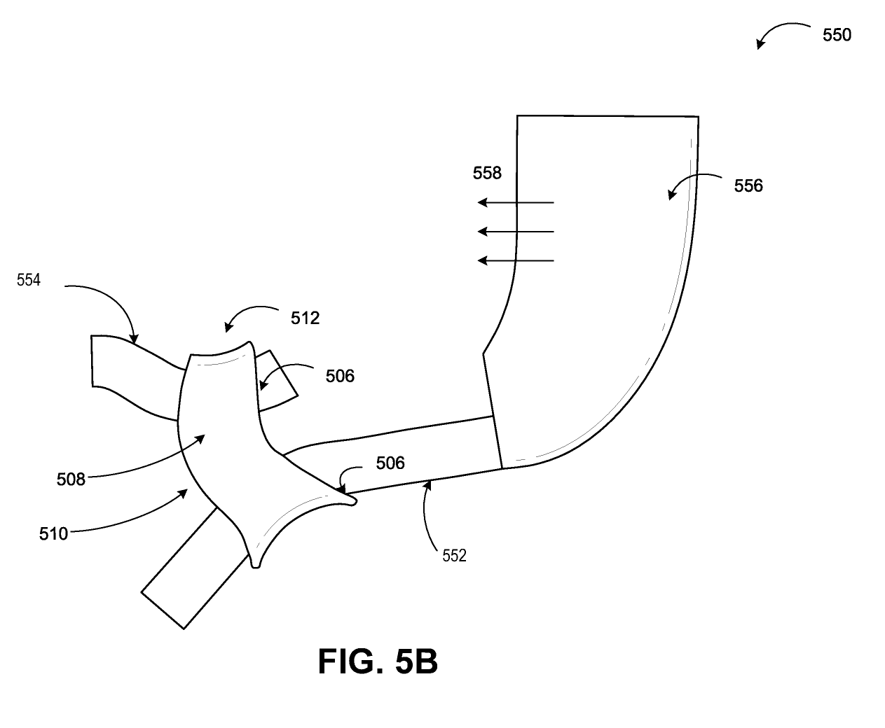

FIGS. 5A and 5B illustrate an example configuration of a meniscal repositioning device configured to bridge anterior or posterior meniscal roots of the menisci, arranged in accordance with at least some embodiments as described herein.

As illustrated in FIG. 5A, diagram 500, a meniscal repositioning device configured to alter a path of more than one meniscal root includes a shortening component 504. The shortening component includes at least two semi-circular channels 506 and a connecting element 508 between the at least two semi-circular channels 506 to enable bridging of an anterior meniscal root of a MM and an anterior meniscal root of a LM. Alternately, a posterior meniscal root of the MM and a posterior meniscal root of the LM may be bridged. Inferior surfaces of the semi-circular channels 506 may exhibit protrusions to increase both a surface area and purchase of the meniscal repositioning device onto the meniscal roots. In some examples, the inferior surfaces of the semi-circular channels 506 are highly polished with a low coefficient of friction. In other examples, the inferior surfaces may exhibit a roughened or porous surface to facilitate soft tissue ongrowth and ingrowth to further promote the mechanical and physical integration of the meniscal repositioning device with the meniscal roots.

The shortening component 504 may be configured to alter the path of at least a portion of the meniscus, which may include the meniscal roots, as the meniscal roots pass through the semi-circular channels 506. As the meniscal roots pass through the semi-circular channels, the functional length of the meniscal roots is reduced such that at least a partial unloading of the ipsilateral tibiofemoral articulation is achieved. By reducing the functional length of the meniscal roots, the at least one portion of the meniscus may be drawn inward towards a midline of a knee to induce a distraction force in one of a medial compartment and a lateral compartment of the knee between the LM and the MM enabling the partial unloading of the ipsilateral tibiofemoral articulation. The shortening component 504 may include contoured outer surfaces 510 such that articulation with a lateral and/or medial femoral condyle may be smooth. Additionally, a curvature 512 of the shortening component may negotiate an insertion of an anterior cruciate ligament (ACL) onto a tibia inferior to the meniscal positioning device.

The shortening component 504 may be applied to the anterior meniscal root of the MM such that the shortening component 504 is affixed to the anterior meniscal root of the LM through a fixation component. Alternately, the shortening component 504 may be applied to the posterior meniscal root of the MM such that the shortening component 504 is affixed to the posterior meniscal root of the LM. Anchorage to a tibial plateau of a tibia, inferior to the meniscal repositioning device, may not be necessary, and thus may potentially remove the need to perforate and/or penetrate the tibial plateau. Manipulation of the wound healing response to mechanically and physically integrate the meniscal repositioning device with the meniscal roots via fibrous tissue proliferation may further remove the need to obtain anchorage to the tibial plateau. Although, using the meniscal repositioning device in conjunction with a suture anchor implanted into the tibial plateau may promote fixation where polished inferior surfaces of the at least two semi-circular channels 506 are used.

As previously discussed, the meniscal repositioning device described in diagram 500 may be delivered arthroscopically and may be of a fixed length, requiring that a surgeon place the meniscal repositioning device over and engage both the anterior meniscal roots of the MM and LM or both the posterior meniscal roots of the MM and LM simultaneously. A range of different sizes of the meniscal repositioning device may be provided to accommodate individual patient anatomy and an integrity and/or state of the menisci.

In another embodiment, a length of the connecting element 508, and therefore an amount of inwards meniscal shifting, may be altered by the surgeon intra-operatively to the amount required to ensure adequate inwards shifting of the MM and/or LM. Such an effect may be achieved if the connecting element 508 is comprised, for example, of multiple individual members, such as a four-bar linkage (i.e., a jack) or other plastically deformable material, or comprised of a shape memory alloy (SMA) where a pre-determined length change may be realized following activation of the meniscal repositioning device. Alternately, the length of the meniscal repositioning device may be changed using a mechanical mechanism having a thread or a ratchet, for example. Furthermore, the angle of the connecting element 508 between the at least two semi-circular channels 506 may also be modified intraoperatively, allowing for the accommodation of individual patient anatomy.

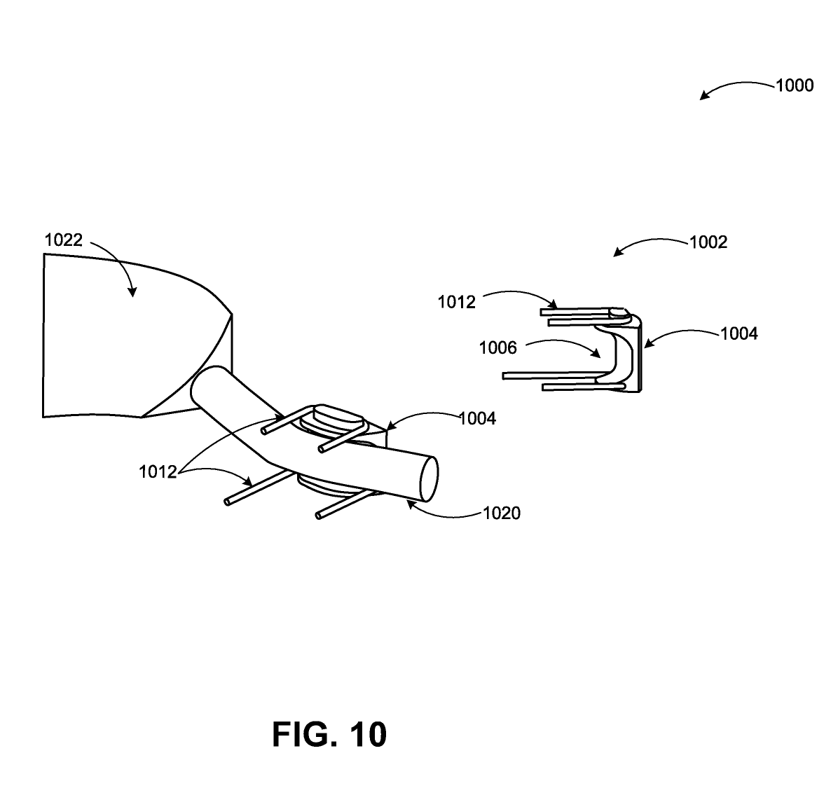

In additional embodiments, the connecting element 508 may be elastic, such that a constant traction force is applied to the bridged anterior meniscal roots of the MM and LM or the bridged posterior meniscal roots of the MM and LM, respectively. Furthermore, the anterior meniscal roots of the MM and LM or the posterior meniscal roots of the MM and LM may be further bridged by a loop of non-degradable suture. A length of the loop governs the amount of meniscal shift, where the loop length may be adjusted intraoperatively and secured using a crimp or other similar elements. The loop, and accordingly the connecting element 508, may also be established between other structures within the medial and/or lateral compartment of the knee, such as between the ACL and the anterior meniscal root of the MM, for example. In further examples, the anterior roots of the MM and LM or the posterior roots of the MM and LM may be either internal to the loop of suture, or external when used in combination with hook-type elements which may bridge the anterior roots. When external and used in combination with hook-type elements, there may be single loop or two loops between the anterior or posterior roots of the MM and LL, inferior and superior suture loops, discussed further in conjunction with FIG. 10. The one or more loops may also be adjusted intraoperatively, and each loop may be adjusted independently.

FIG. 5B, diagram 550, illustrates a superior view of the meniscal repositioning device configured to alter a path of an anterior meniscal root of the LM 554 and an anterior root of the MM 552. As discussed above in conjunction with FIG. 5A, the shortening component may include at least two semi-circular channels 506 and the connecting element 508 between the semi-circular channels 506 to enable mating of the anterior meniscal root of the LM 554 and the anterior root of the MM 552. The shortening component may be configured to alter the path of at least a portion of the MM 556 (illustrated) and/or the LM, which may include the anterior meniscal roots of the MM 552 and/or the anterior meniscal roots of the LM 554, as the anterior meniscal roots pass through the semi-circular channels 506. As the anterior meniscal roots pass through the semi-circular channels 506, a functional length of the anterior meniscal roots 552, 554 may be reduced such that at least a partial unloading of the ipsilateral tibiofemoral articulation is achieved. By reducing the functional length of the meniscal roots, the at least one portion of the MM 556 and/or LM may be drawn inward 558 towards a midline of a knee to induce a distraction force in a medial compartment and/or a lateral compartment of the knee between the LM and the MM enabling the partial unloading of the ipsilateral tibiofemoral articulation. The shortening component 504 may further include contoured outer surfaces 510 such that articulation with a femoral condyle may be smooth. Additionally, a curvature 512 of the shortening component may negotiate an insertion of an anterior cruciate ligament (ACL) onto a tibia inferior to the meniscal positioning device.

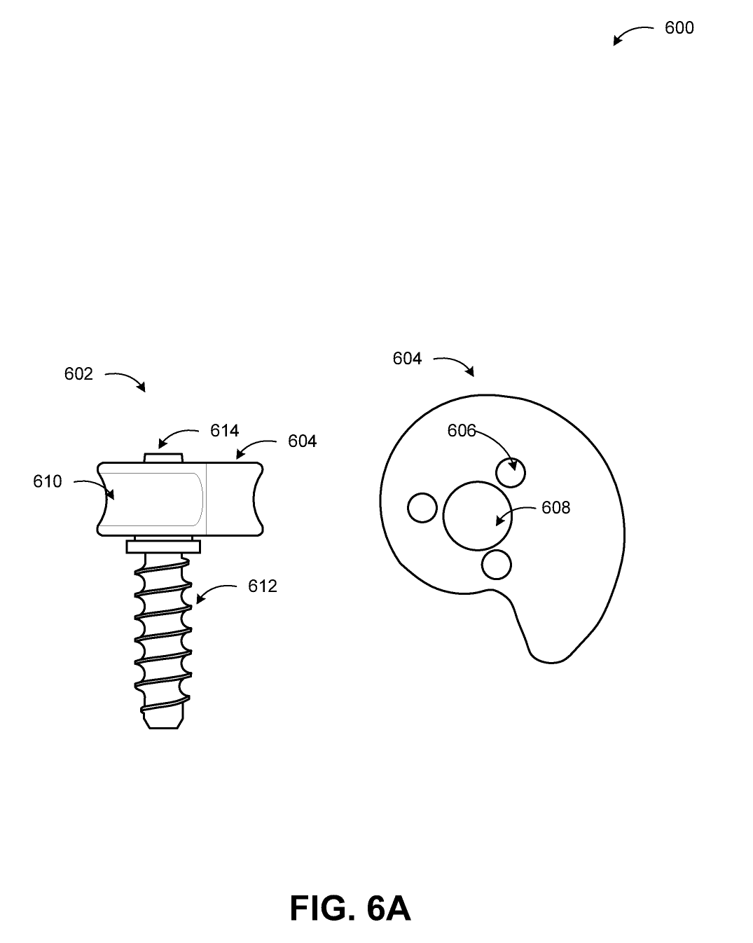

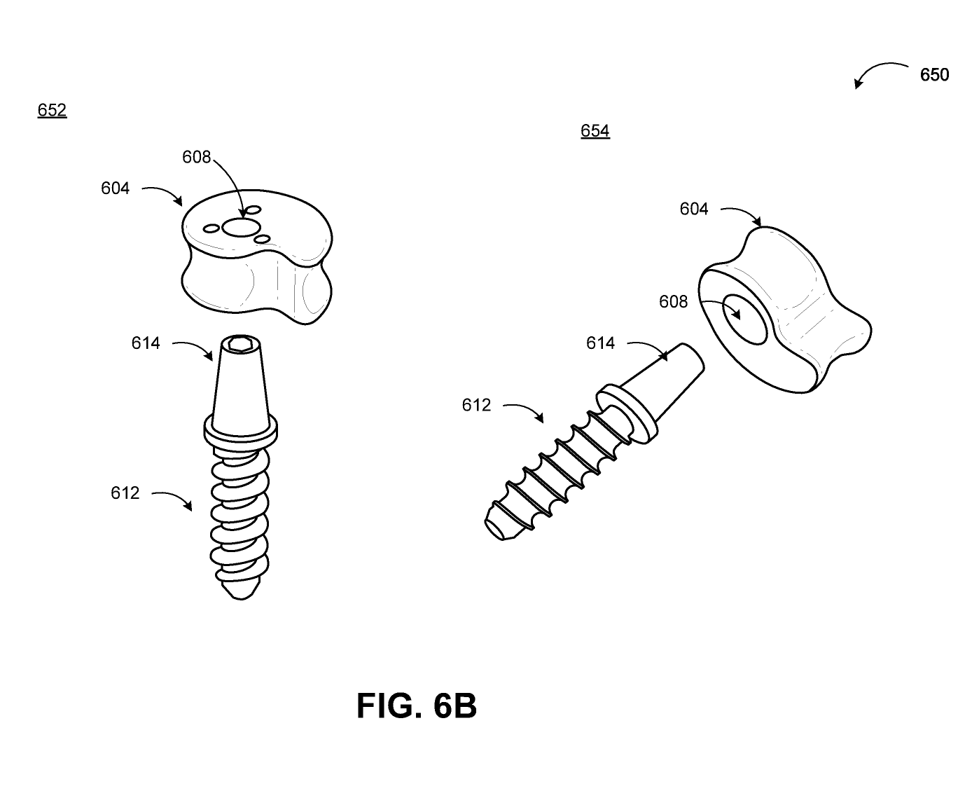

FIGS. 6A and 6B illustrate an example configuration of a meniscal repositioning device as a mechanical cam, arranged in accordance with at least some embodiments as described herein.

As shown in FIG. 6A, diagram 600, a meniscal repositioning device 602 may include a shortening component 604 resembling a mechanical cam, which may be symmetrical or asymmetrical, and a fixation component 612 comprising a bone screw. The shortening component 604 may include a curved channel 610 on an exterior surface of the shortening component 604 configured to accommodate a passage of a meniscal root of a meniscus, where the meniscus may be a LM or a MM, and the meniscal root may be an anterior or posterior meniscal root. In some embodiments, the curved channel 610 accommodating the passage of the meniscal root may trace a helical or other path. In other embodiments, the exterior surface of the shortening component 604 configured to accommodate a passage of a meniscal root of a meniscus may exhibit a flattened profile. Furthermore, while a spiral-shaped shortening component 604 is illustrated, several other shape profiles may be possible for the shortening component 604. For example, the shortening component 604 may be constructed like a pie, where segments which are not required may be removed. A weight of the shortening component 604 may also be reduced by introducing selected perforations to remove excess material.

The shortening component 604 may include a mating female Morse taper 608 to facilitate fixation of the shortening component 604 to a tibia through the fixation component 612. The shortening component 604 may further include two or more recesses 606 for mating with a driver to enable mechanical rotation of the shortening component 604. A working distance of the shortening component 604 may be adjusted to an optimal amount by the surgeon intra-operatively through rotation of the shortening component 604 employing the driver. The rotation may facilitate altering the path of at least one portion of the meniscus, which may include the meniscal root, as the meniscal root passes through the curved channel 610. As the meniscal root passes through the curved channel 610, the functional length of the meniscal root may be reduced, such that the meniscus is drawn inward towards a midline of the knee to induce a distraction force in a medial or lateral compartment of the knee between MM and LM achieving at least a partial unloading of the ipsilateral tibiofemoral articulation.

The fixation component 612 may include a cancellous and/or cortical threaded bone screw for anchorage of the shortening component 604 into a tibial plateau of a tibia inferior to the meniscal repositioning device 602. In some examples, the screw may only be a single piece or may be comprised of two or more pieces, such as the co-linear threads and a male Morse taper 614, as illustrated, which may be assembled in a sequential fashion in situ or external to the knee further limiting a size of an arthroscopic portal required. In other embodiments, axes of the threads and the male Morse taper 614 may be at some angle other than 0°, such that they are not co-linear. The threads may be right-handed threads or left-handed threads, where the left-handed threads may be employed to minimize potential of loosening of the screw with torque loading. Another screw or component may be used in combination with the screw to further limit this potential.

The fixation component 612 may include the male Morse taper 614 at a proximal end of the fixation component 612, where the male Morse taper 614 of the fixation component 612 may mate with the female Morse taper 608 of the shortening component 604. The male Morse taper 614 of the fixation component 612 may further be configured to permit relative rotation of the shortening component 604 to obtain the desired path alteration of the at least one portion of the meniscus. In some examples, the male Morse taper 614 of the fixation component 612 may be further configured to permit a locking mechanism to fix the shortening component 604 in a particular position, wherein the locking mechanism may be a reverse taper at the proximal end of the fixation component 612. The locking mechanism may be employed once the shortening component 604 has been rotated to obtain the desired path alteration. In some examples, the screw may be integrated with the male Morse taper 614 via a multiaxial joint, which is locked into position mechanically. In other embodiments, nuts or other similar mechanical devices may be used as a locking mechanism.

In other examples, a ratchet system may be employed, which may negate the need for a mating female and male Morse taper, perhaps reducing the design to a single component delivered by a surgeon. For example, the meniscal repositioning device may be a single component with no Morse taper or similar junction. That is, the device may be essentially a screw with a head shaped as a mechanical cam.

To implant the meniscal repositioning device 602, the screw of the fixation component 612 may be introduced into a pilot hole created using a drill, or through self-tapping of the screw. In some examples, a stepped drill may be used to provide a planar surface for seating of the screw onto the bone via a shoulder incorporated into the screw design. While a planar surface has been used to illustrate the tibial plateau surface in previous figures, it is appreciated that the surface of the tibial plateau may be highly uneven and variable. Accordingly, the implanted screw may not necessarily lie in a plane perpendicular to the meniscus. A relative position and distance of the pilot hole from the meniscal root may be governed by accompanying surgical instruments. The distance may allow the passing of the shortening component 604 over the screw of the fixation component 612 at a smallest radial working distance. Once the shortening component 604 is engaging both the screw of the fixation component 612 and the meniscal root, a driver may be introduced to enable rotation of the shortening component 604.

As illustrated, the shortening component 604 has a series of 3 cylindrical recesses 606 at equal radial distances from a central axis and separated by 120°, which may be engaged by the driver through a series of cylindrical prongs on the driver. Employing the driver, the surgeon may rotate the shortening component 604, thereby causing an alteration in the path taken by the at least one portion of the meniscus, which may include the meniscal root, as the meniscal root passes through the curved channel 610. As the meniscal root passes through the curved channel 610, the rotation may cause a reduction in a functional length of the meniscal root, such that the meniscus is drawn inward towards a midline of the knee to induce a distraction force in a medial or lateral compartment of the knee between MM and LM achieving at least a partial unloading of the ipsilateral tibiofemoral articulation. The amount of the inwards meniscal shift in the above-described embodiment may be governed by the pilot hole and screw position, and the relative rotation and working distance of the shortening component 604.

Once the surgeon has introduced a desired amount of meniscal displacement, a series of axially-directed blows may be applied to the driver and shortening component 604 to lock the male Morse taper 614 into that position of relative rotation. In another embodiment, a second surgical procedure could be performed to unlock the male Morse taper 614 and induce additional and further meniscal shifting.

FIG. 6B, diagram 650, illustrates a superior aspect 652 and an inferior aspect 654 of the mating of the male Morse taper 614 at the proximal end of the screw comprising the fixation component 612 and the female Morse taper 608 of the shortening component 604.

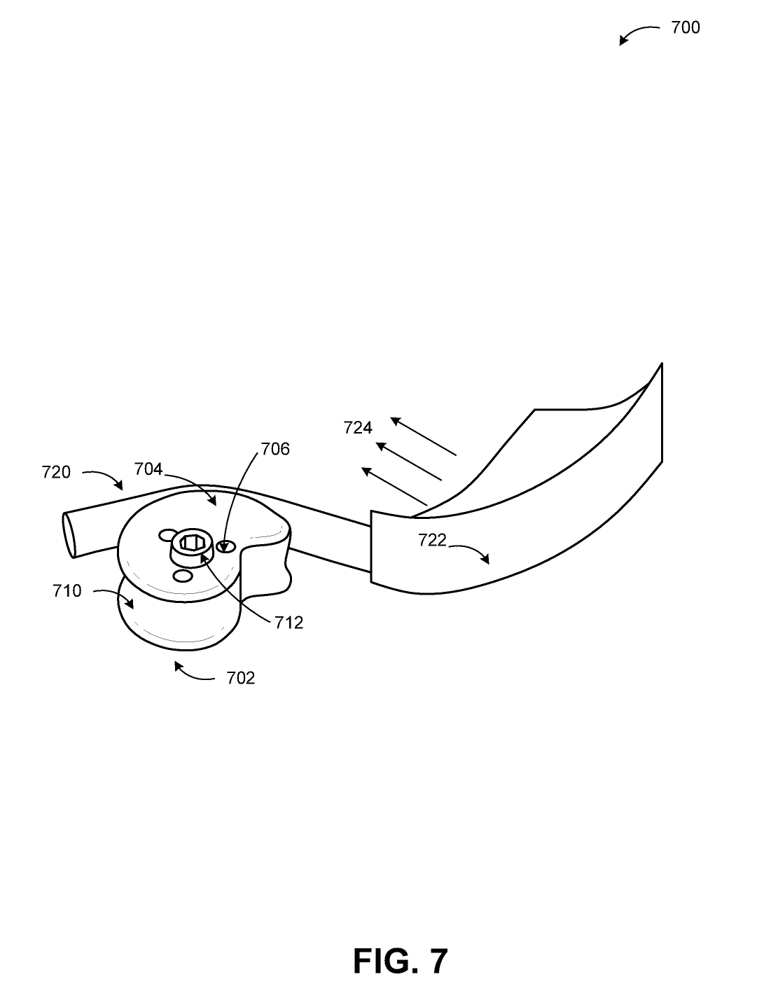

FIG. 7 illustrates an altered path of a meniscal root employing a mechanical cam meniscal repositioning device, arranged in accordance with at least some embodiments as described herein.

As shown in diagram 700, a meniscal repositioning device 702 may include a shortening component 704 resembling a mechanical cam and a fixation component 712 comprising a screw engaged with the shortening component 704. The shortening component 704 may include a curved channel 710 on an exterior surface of the shortening component 704 configured to accommodate a passage of a meniscal root 720 of a meniscus 722, where the meniscus may be a LM or a MM, and the meniscal root may be an anterior or posterior meniscal root. The shortening component 704 may include a mating female Morse taper to facilitate fixation of the shortening component 704 to a tibia through the fixation component 712. The shortening component 704 may further include two or more recesses 706 for mating with a driver to enable mechanical rotation of the shortening component 704. A working distance of the shortening component 704 may be adjusted to an optimal amount by a surgeon intra-operatively through rotation of the shortening component 704 employing the driver. The rotation may facilitate altering the path of at least one portion of the meniscus, which may include the meniscal root 720, as the meniscal root 720 passes through the curved channel 710. As the meniscal root 720 passes through the curved channel 710, the rotation may cause a functional length of the meniscal root 720 to be reduced, such that the meniscus 722 is drawn inward 724 towards a midline of the knee to induce a distraction force in a medial or lateral compartment of the knee between MM and LM, achieving at least a partial unloading of the ipsilateral tibiofemoral articulation.

The fixation component 712 may include a cancellous and/or cortical threaded bone screw for anchorage of the shortening component 704 into a tibial plateau of the tibia inferior to the meniscal repositioning device 702. The fixation component 612 may include a male Morse taper at a proximal end of the fixation component 712, where the male Morse taper of the fixation component 712 may mate with the female Morse taper of the shortening component 704 to engage the shortening component 704 and the fixation component 712, as illustrated. The male Morse taper of the fixation component 712 may further be configured to permit relative rotation of the shortening component 704 to obtain the desired path alteration of the at least one portion of the meniscus employing the driver. In other examples, the male Morse taper of the fixation component 712 may be further configured to permit a locking mechanism to fix the shortening component 704 in a particular position, wherein the locking mechanism may be a reverse taper at the proximal end of the fixation component 712. A series of axially-directed blows may be applied to the driver and shortening component 704 to lock the male Morse taper 614 into that position of relative rotation. The locking mechanism may be employed once the shortening component 704 has been rotated to obtain the desired path alteration. In another embodiment, a second surgical procedure could be performed to unlock the male Morse taper and induce additional and further meniscal shifting. The adjustment function in a subsequent surgical procedure may be further promoted through the use of a ratchet mechanism positioned between the shortening component 704 and the fixation component 712.

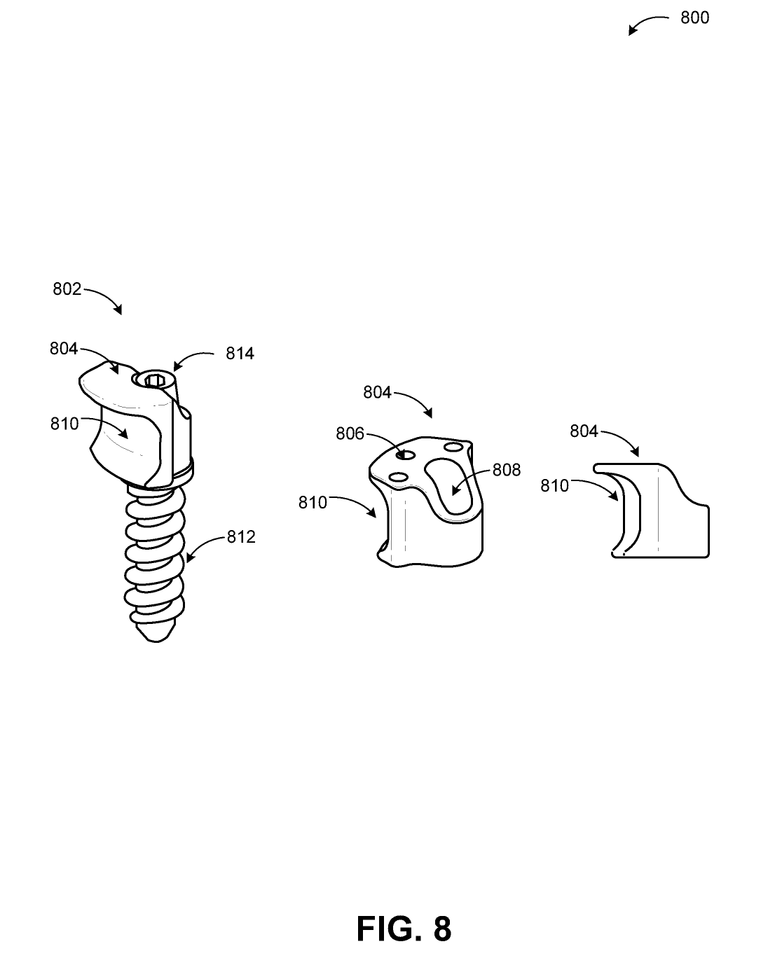

FIG. 8 illustrates an example configuration of a meniscal repositioning device as a condensed mechanical cam, arranged in accordance with at least some embodiments as described herein.

Similar to the meniscal repositioning device discussed in conjunction with FIG. 7, a meniscal repositioning device 802 may include a shortening component 804 resembling a mechanical cam and a fixation component 812 comprising a bone screw engaged with the shortening component 804. However, the shortening component 804 may resemble a condensed mechanical cam, as illustrated in diagram 800. The shortening component 804 may include a curved channel 810 on an exterior surface of the shortening component 804 configured to accommodate a passage of a meniscal root of a meniscus, where the meniscus may be a LM or MM, and the meniscal root may be an anterior or posterior meniscal root. The shortening component 804 may include a mating female Morse taper 808 to facilitate fixation of the shortening component 804 to a tibia through the fixation component 812. The shortening component 804 may further include two or more recesses 806 for mating with a driver to enable mechanical rotation of the shortening component 804. A working distance of the shortening component 804 may be adjusted to an optimal amount by a surgeon intra-operatively through rotation of the shortening component 804 employing the driver. The rotation may facilitate altering the path of at least one portion of the meniscus, which may include the meniscal root, as the meniscal root passes through the curved channel 810. As the meniscal root passes through the curved channel 810, the rotation may cause a reduction in a functional length of the meniscal root, such that the meniscus is drawn inward towards a midline of the knee to induce a distraction force in a medial or lateral compartment of the knee between MM and LM, achieving at least a partial unloading of the ipsilateral tibiofemoral articulation.

The fixation component 812 may include a cancellous and/or cortical threaded bone screw for anchorage of the shortening component 804 into a tibial plateau of the tibia inferior to the meniscal repositioning device 802. The fixation component 812 may include a male Morse taper 814 at a proximal end of the fixation component 812, where the male Morse taper 814 of the fixation component 812 may mate with the female Morse taper 808 of the shortening component 804 to engage the shortening component 804 and the fixation component 812, as illustrated. The male Morse taper 814 of the fixation component 812 may further be configured to permit relative rotation of the shortening component 804 to obtain the desired path alteration of the at least one portion of the meniscus employing the driver such that the meniscus is drawn inward towards a midline of the knee to induce a distraction force in a medial or lateral compartment of the knee between MM and LM achieving at least a partial unloading of the ipsilateral tibiofemoral articulation.

To implant the meniscal repositioning device 802 in the above-described embodiment, the meniscal root may be released and shifted slightly allowing implantation of the screw comprising the fixation component 812. The shortening component 804 may then be introduced and rotated to a desired position employing the driver, with the final relative position secured using a series of axially-directed blows delivered by a surgeon. In another embodiment, the screw comprising the fixation component 812 may be at a predefined angle to the shortening component 804, which may benefit implantation. In a further embodiment, the junction between the screw comprising the fixation component 812 and the shortening component 804 may be modelled as a ball and socket joint, permitting three rotational degrees of freedom and secured using a set-screw of various descriptions to improve the delivery and implantation of the meniscal repositioning device 802.

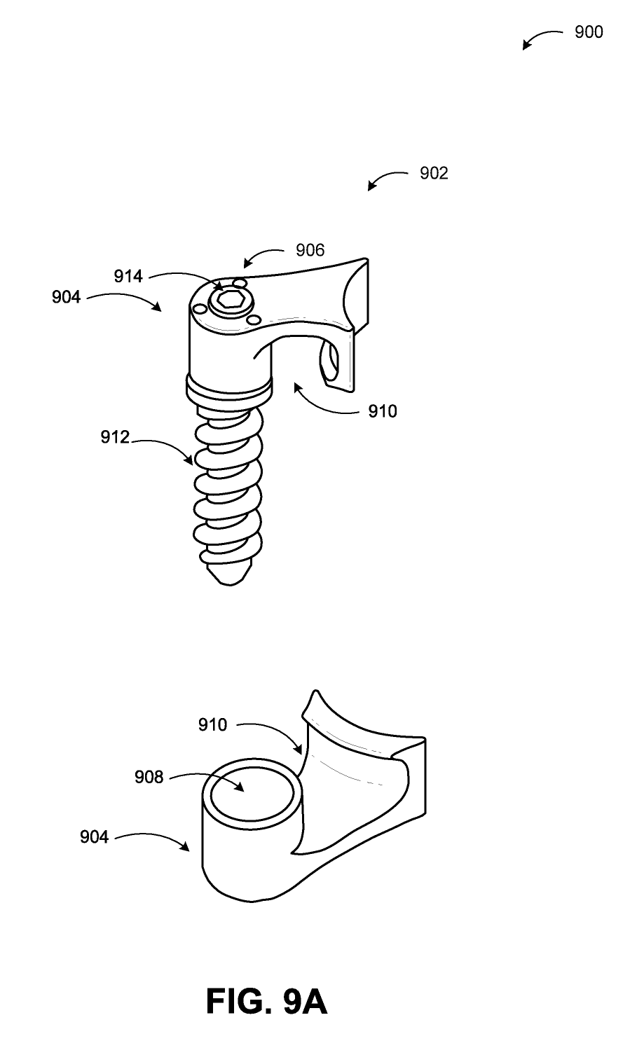

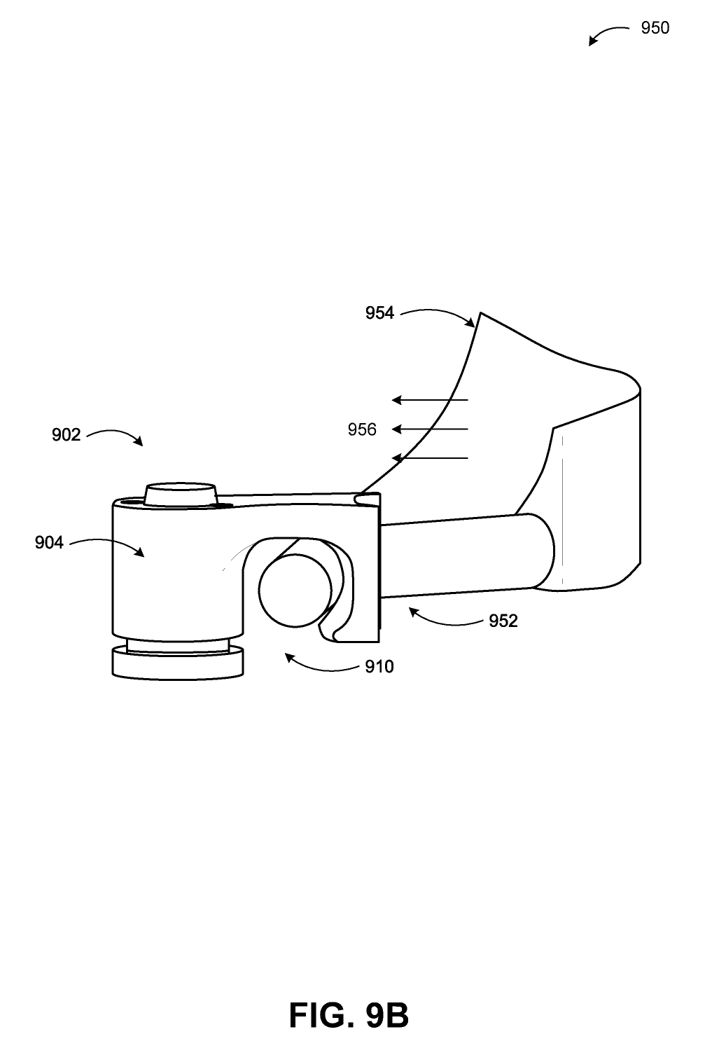

FIGS. 9A and 9B illustrate another example configuration of a meniscal repositioning device, arranged in accordance with at least some embodiments as described herein.

As shown in FIG. 9A, diagram 900, a meniscal repositioning device 902 may include a shortening component 904 employed in combination with a fixation component 912 comprising a bone screw. The shortening component 904 may be shaped to pass over and/or beneath a meniscal root of a meniscus and, where the meniscus may be a MM or a LM, and the meniscal root may be an anterior or posterior meniscal root. The shortening component 904 may include an appropriately contoured channel 910 on an interior surface of the shortening component 904 to accommodate a passage of the meniscal root. The shortening component 904 may also include a mating female Morse taper 908 to facilitate fixation of the shortening component 904 to a tibia through the fixation component 912. The shortening component 904 may further include two or more recesses 906 for mating with a driver to enable mechanical rotation of the shortening component 904. A working distance of the shortening component 904 may be adjusted to an optimal amount by a surgeon intra-operatively through rotation of the shortening component 904 employing the driver. The rotation may facilitate the path alteration of the portion of the meniscus, which may include the meniscal root, as the meniscal root passes through the contoured channel 910. As the meniscal root passes through the contoured channel 910, the rotation may cause a reduction in a functional length of the meniscal root, such that the meniscus is drawn inward towards a midline of the knee to induce a distraction force in a medial or lateral compartment of the knee between MM and LM, achieving at least a partial unloading of the ipsilateral tibiofemoral articulation.

The fixation component 912 may include a cancellous and/or cortical threaded bone screw for anchorage of the shortening component 904 into a tibial plateau of the tibia inferior to the meniscal repositioning device 902. The fixation component 912 may include a male Morse taper 914 at a proximal end of the fixation component 912, where the male Morse taper 914 of the fixation component 912 may mate with the female Morse taper 908 of the shortening component 904 to engage the shortening component 904 and the fixation component 912, as illustrated. The male Morse taper 914 of the fixation component 912 may further be configured to permit relative rotation of the shortening component 904 to obtain the desired path alteration of the at least one portion of the meniscus employing the driver.

FIG. 9B, diagram 950, illustrates the meniscal repositioning device 902 comprising the shortening component 904 employed in combination with the fixation component 912, the shortening component 904 shaped to pass over a meniscal root 952 of a meniscus 954. As previously discussed, the shortening component 904 includes a contoured channel 910 to accommodate a passage of the meniscal root 952. The working distance of the shortening component 904 may be adjusted to an optimal amount by a surgeon intra-operatively through rotation of the shortening component 904 employing a driver. The rotation may facilitate the path alteration of the portion of the meniscus 954, which may include the meniscal root, as the meniscal root 952 passes through the contoured channel 910. As the meniscal root passes through the contoured channel 910, the rotation may cause a reduction in a functional length of the meniscal root 952, such that the meniscus 954 is drawn inward 956 towards a midline of the knee to induce a distraction force in a medial or lateral compartment of the knee between MM and LM, achieving at least a partial unloading of the ipsilateral tibiofemoral articulation.

To implant the above-described meniscal repositioning device 902, which may likely be an arthroscopic, minimally-invasive procedure, the screw of the fixation component 912 may be implanted, down to a level of a shoulder incorporated into the screw design. The shortening component 904 may then be fitted over, as illustrated, and/or under the meniscal root 952 to engage, and a suitable amount of shift and/or force may be applied to induce a deviation of the meniscal root 952 as well as to engage the male Morse taper 914 of the fixation component 912 discussed in conjunction with FIG. 9A. Once seated, but not secured, the driver may be employed to apply additional torque if needed to the meniscal repositioning device 902, which would induce further deviation of the meniscal root 952. Once the desired amount of deviation has been achieved, several blows may be delivered to the driver and the meniscal repositioning device 902 to lock the male Morse taper 914 into the current position.

FIG. 10 illustrates an example configuration of a meniscal repositioning device including sutures for fixation, arranged in accordance with at least some embodiments as described herein.

As shown in diagram 1000, a meniscal repositioning device 1002 may include a shortening component 1004 comprising a semi-circular channel 1006 on an interior surface of the shortening component 1004, a fixation component, and one or more inferior and superior suture loops 1012 facilitating integration of the shortening component 1004 and the fixation component. Anchorage of the meniscal repositioning device 1002 into a tibia adjacent to a meniscal root 1020 of a meniscus 1022 may be achieved using the fixation component, where the fixation component may be a screw, a post and/or a suture anchor, among other components. Alternatively, anchorage could be achieved using a bar which may passed through subchondral bone via a drill hole and flipped. In either embodiment, the shortening component 1004 may be integrated with the fixation component using the inferior and superior suture loops 1012, which may be adjustable in length. Accordingly, the shortening component 1004 may be passed beneath the meniscal root 1020 following anchorage or assembled over the meniscal root 1020. In the above embodiment, the path alteration of the meniscal root 1020 induced by the meniscal repositioning device 1002 may be limited to the axial plane.

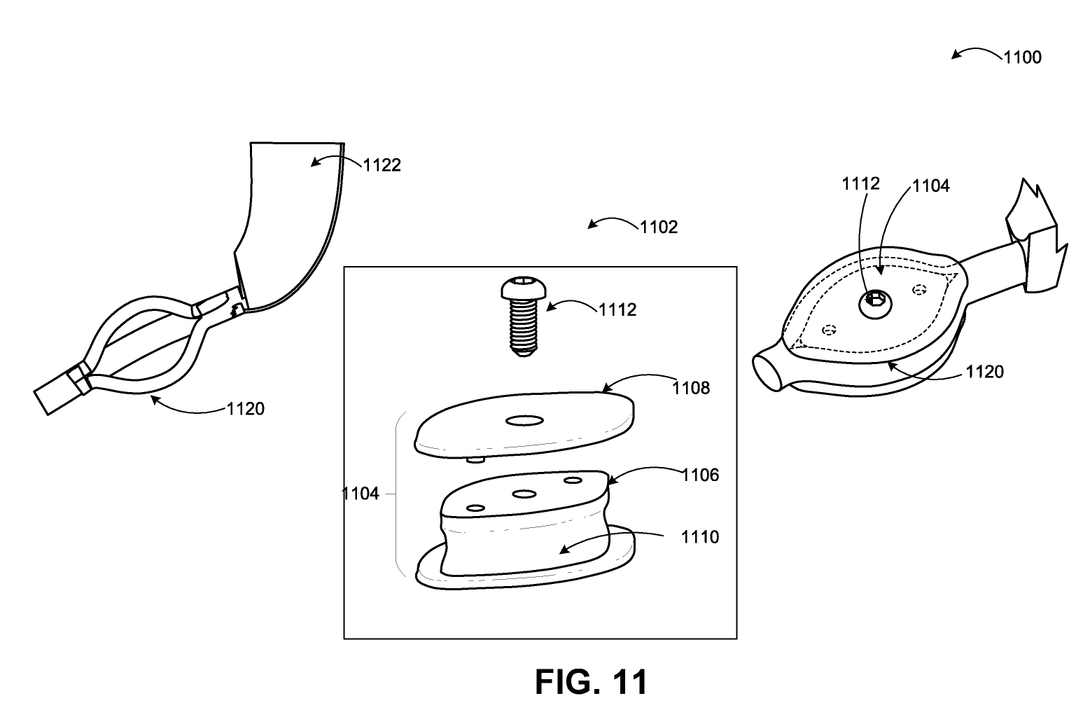

FIG. 11 illustrates an example configuration of a meniscal repositioning device inserted in a bisected meniscal root, arranged in accordance with at least some embodiments as described herein.

As shown in diagram 1100, a meniscal root of a meniscus 1122 may be dissected along a length of the meniscal root into two bundles forming a bisected meniscal root 1120, where a blunt dissection of parallel-fibred collagenous tissue is preferable. A gap created by the bisected meniscal root 1120 may be filled by a meniscal repositioning device 1102, the shape of which causes an alteration of the two bundles of the bisected meniscal root 1120 from their normal path. The alteration of the path may effectively reduce the functional length of the bisected meniscal root 1120, such that the meniscus 1122 is drawn inward towards a midline of the knee to induce a distraction force in a medial or lateral compartment of the knee between MM and LM, achieving at least a partial unloading of the ipsilateral tibiofemoral articulation.

The meniscal repositioning device 1102 may include a shortening component 1104 and a fixation component 1112, the assembled meniscal repositioning device 1102 resembling a spool squashed in one-dimension. The shortening component may include an inner component 1106 and an outer component 1108. The inner component 1106 may include a curved channel 1110 on an exterior surface of the inner component 1106 configured to accommodate a passage of the two bundles of the bisected meniscal root 1120. The outer component 1108 may act as a cover positioned over the inner component, which is screwed into place using the fixation component 1112.

In another embodiment, the meniscal repositioning device may be a tear-drop shape and may be symmetrical or asymmetrical. The two bundles of the bisected meniscal root may be formed by teasing apart fibers comprising the meniscal root, and the meniscal repositioning device may be inserted in the gap between the two bundles. In a further embodiment, the meniscal repositioning device may include proximal and distal rings positioned over the bisected meniscal root to limit effects of a transverse component of force created by the introduced alteration to the path of the two bundles of the bisected meniscal root.

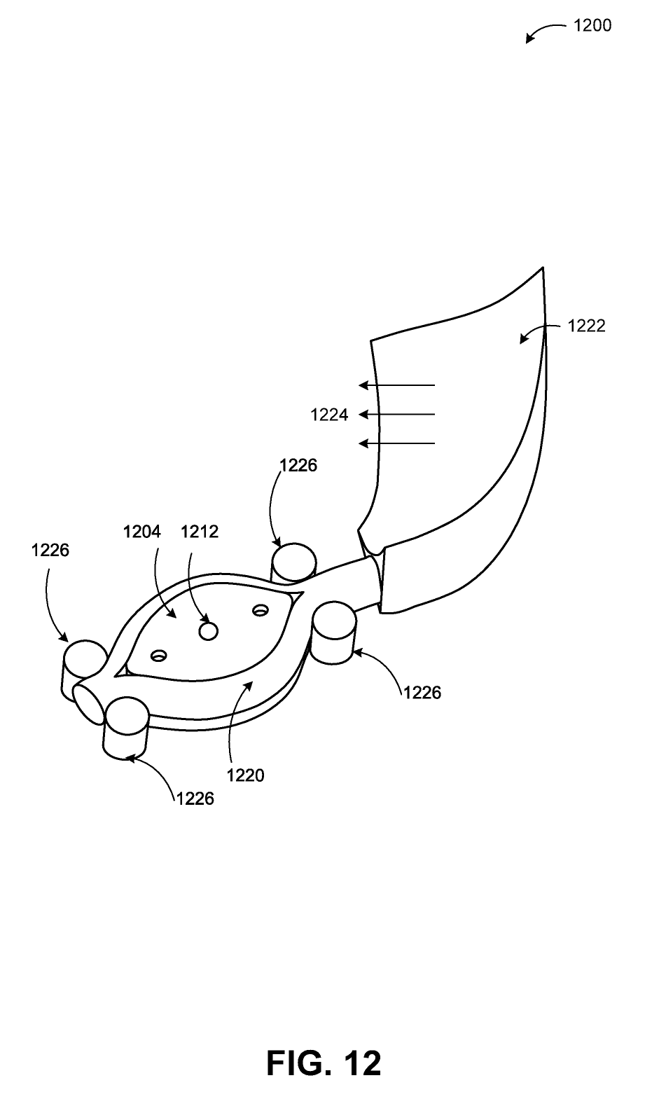

FIG. 12 illustrates an example configuration of a meniscal repositioning device including support members inserted in a bisected meniscal root, arranged in accordance with at least some embodiments as described herein.

As previously discussed in conjunction with FIG. 11, a meniscal root of a meniscus 1222 may be dissected along a length of the meniscal root into two bundles forming a bisected meniscal root 1220, as illustrated in diagram 1200. A gap created by the bisected meniscal root 1220 may be filled by a meniscal repositioning device, the shape of which causes an alteration of the two bundles of the bisected meniscal root 1220 from their normal path. The alteration of the path may effectively reduce the functional length of the bisected meniscal root 1220, such that the meniscus 1222 is drawn inward 1224 towards a midline of the knee to induce a distraction force in a medial or lateral compartment of the knee between MM and LM, achieving at least a partial unloading of the ipsilateral tibiofemoral articulation.

The meniscal repositioning device may include a shortening component 1204, a fixation component 1212, and one or more support members 1226. The shortening component may include an inner component comprising a curved channel on an exterior surface of the inner component configured to accommodate a passage of the two bundles of the bisected meniscal root 1220. The shortening component may also include an outer component acting as a cover and positioned over the inner component, which is screwed into place using the fixation component 1212, which further facilitates fixation with a portion of a tibia adjacent to the bisected meniscal root 1220. The support members 1226 may be configured to support an exterior portion of the bisected meniscal root 1220. The support members 1226 may be further configured to counter a transverse component of force created by the alteration to the path of the two bundles of the bisected meniscal root 1220. The effect of this force may be further minimized if a roughened and or porous texture was utilized on the surface, which would facilitate the physical and mechanical integration of the device with the meniscal root through fibroproliferation. An osteoconductive surface may be employed on an inferior surface of the shortening component 1204 to promote bone growth to facilitate fixation with the portion of the tibia adjacent to the bisected meniscal root 1220.

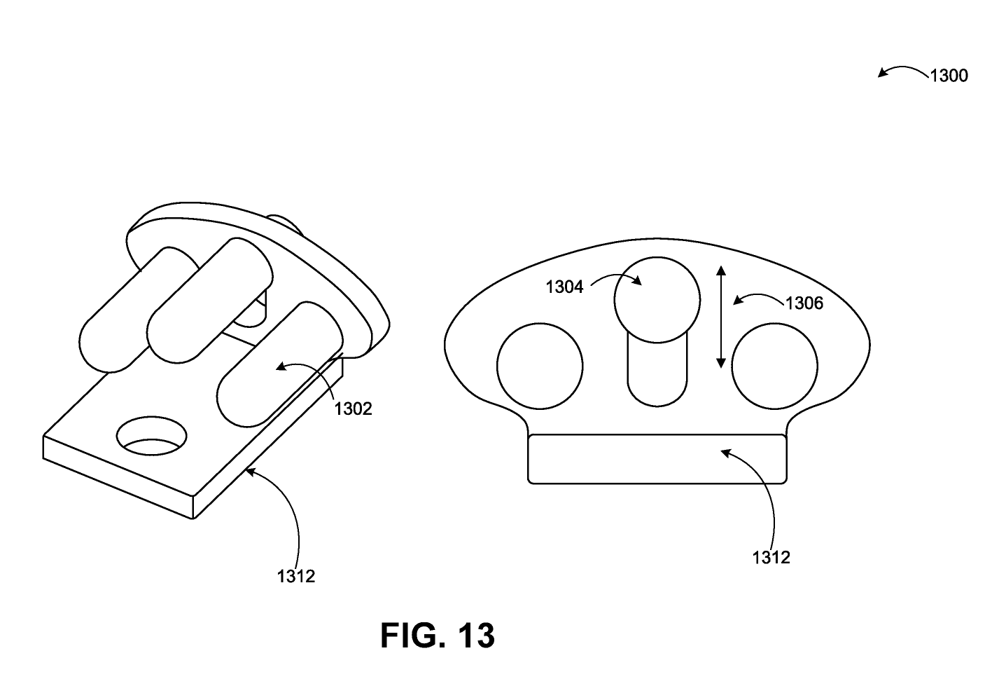

FIG. 13 illustrates an example configuration of a meniscal repositioning device including movable prongs, arranged in accordance with at least some embodiments as described herein.

As shown in diagram 1300, a meniscal repositioning device may include at least three prongs 1302 extending laterally from a shortening component 1312. At least one of the prongs, such as prong 1304 may be movable. The prongs may translate in a forward and backward motion as illustrated by arrow 1306 to effectively adjust a position of the at least one prong 1304. Adjusting a position of the prong 1304 may intra-operatively enable a surgeon to adjust a functional length of a meniscal root of a meniscus, where a passage of the meniscal root may be accommodated by the shortening component 1312. Reducing the functional length of the meniscal root may cause the meniscus to be drawn inward towards a midline of the knee to induce a distraction force in a medial or lateral compartment of the knee between a MM and a LM, achieving at least a partial unloading of an ipsilateral tibiofemoral articulation. In another embodiment, the pronged device may be applied to the meniscal root and not be fixed to the proximal tibia, and be free to translate with the meniscal root. In a further embodiment, the distal ends of the prongs could exhibit geometry to provide a latching action and promote fixation to the meniscal root.

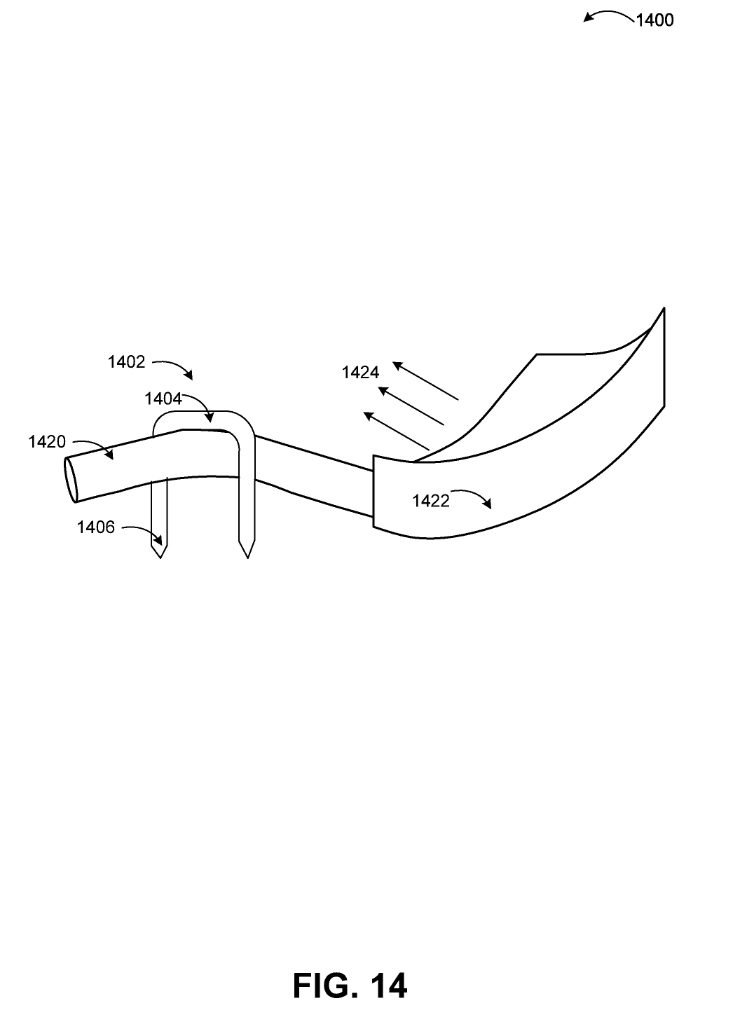

FIG. 14 illustrates an altered path of a meniscal root employing a staple as a meniscal repositioning device, arranged in accordance with at least some embodiments as described herein.

As shown in diagram 1400, a meniscal repositioning device 1402 may include a shortening component 1404 and a fixation component 1406 integrated into a single structure. The meniscal repositioning device 1402 may be a staple, for example. The shortening component 1404 may include a curved channel on an interior surface of the shortening component 704 configured to accommodate a passage of a meniscal root 1420 of a meniscus 1422, where the meniscus may be a LM or a MM, and the meniscal root may be an anterior or posterior meniscal root. The fixation component 1406 may be configured to affix the meniscal repositioning device 1402 to a tibial plateau of the tibia inferior to the meniscal repositioning device 1402. A working distance of the shortening component 1404 may be adjusted to an optimal amount by a surgeon intra-operatively through rotation of the meniscal repositioning device 1402. The rotation may facilitate altering the path of at least one portion of the meniscus, which may include the meniscal root 1420, as the meniscal root 1420 passes through the curved channel. As the meniscal root 1420 passes through the curved channel, the rotation may cause a functional length of the meniscal root 1420 to be reduced, such that the meniscus 1422 is drawn inward 1424 towards a midline of the knee to induce a distraction force in a medial or lateral compartment of the knee between MM and LM, achieving at least a partial unloading of the ipsilateral tibiofemoral articulation. In some embodiments, the staple may be resorbable.

According to some examples, a meniscal repositioning device may be described. An example meniscal repositioning device may include a shortening component comprising a semi-circular channel on an interior surface of the shortening component, the semi-circular channel configured to accommodate a passage of a meniscal root. The shortening component may be configured to alter a path of at least one portion of a meniscus in order to reduce a functional length of the meniscal root such that at least a partial unloading of the ipsilateral tibiofemoral articulation is achieved. The example meniscal repositioning device may also include a fixation component configured to affix the shortening component to a portion of a tibia adjacent to the meniscal root, an anterior portion of the meniscal root, and/or a posterior portion of the meniscal root.

In other examples, the portion of the meniscus may be drawn inward towards a midline of a knee to induce a distraction force in a medial compartment or a lateral compartment of the knee between a medial meniscus and a lateral meniscus. The shortening component may be substantially elongated such that the meniscal root is enabled to translate in a substantially perpendicular direction with respect to the semi-circular channel. The semi-circular channel may include a series of smooth contours to facilitate path alteration of the at least one portion of the meniscus as the meniscal root passes through the semi-circular channel. A first end and a second end of the semi-circular channel may be in a substantially same horizontal plane.

In further examples, the fixation component may include an anchor component attached to an inferior surface of the semi-circular channel. The anchor component may be configured to enable fixation of the shortening component to the tibia through a bone screw inserted through a corresponding hole in the anchor component. An inferior surface of the anchor component may include a series of prongs to facilitate fixation to the adjacent tibia and/or an osteoconductive surface to promote bone growth to facilitate fixation with the adjacent tibia. The shortening component may include at least two semi-circular channels and a connecting element between the two semi-circular channels to enable bridging of the anterior portion of the meniscal root of a medial meniscus and the anterior portion of the meniscal root of a lateral meniscus and/or the posterior portion of the meniscal root of the medial meniscus and the posterior portion of the meniscal root of the lateral meniscus. The shortening component is applied to the anterior portion of the meniscal root of the medial meniscus such that the shortening component is affixed to the anterior portion of the meniscal root of the lateral meniscus through the fixation component or applied to the posterior portion of the meniscal root of the medial meniscus such that the shortening component is affixed to the posterior portion of the meniscal root of the lateral meniscus through the fixation component. The shortening component may include an adjustable loop of suture formed around or between one of: the anterior portion of the meniscal root of a medial meniscus and the anterior portion of the meniscal root of a lateral meniscus, and the posterior portion of the meniscal root of the medial meniscus and the posterior portion of the meniscal root of the lateral meniscus. The shortening component and fixation component may be integrated into a single structure, where the single structure is a staple.

According to other embodiments, a meniscal repositioning device to achieve unloading of an ipsilateral tibiofemoral articulation may be described. An example meniscal repositioning device may include a shortening component comprising a curved channel on an exterior surface of the shortening component, the curved channel configured to accommodate a passage of a meniscal root. The shortening component may be configured to alter a path of at least one portion of a meniscus in order to reduce a functional length of a meniscal root such that at least a partial unloading of the ipsilateral tibiofemoral articulation is achieved. The example meniscal repositioning device may also include a fixation component configured to affix the shortening component to a portion of a tibia adjacent to the meniscal root.

In other embodiments, the shortening component may include a mating female Morse taper to facilitate fixation of the shortening component to the adjacent tibia through the fixation component, where the fixation component may be a threaded bone screw to facilitate anchorage in the adjacent tibia, and may include a male Morse taper at a proximal end. The male Morse taper of the fixation component may be configured to permit relative rotation of the shortening component to obtain a desired path alteration of the at least one portion of the meniscus. The male Morse taper of the fixation component may be further configured to permit a locking mechanism to fix the shortening component in a particular position, where the locking mechanism may be a reverse taper at a proximal end of the fixation component.

The shortening component may include two or more recesses for mating with a driver to enable mechanical rotation of the shortening component. The fixation component may include a series of sutures accommodated by a plurality of recesses in the shortening component configured to stabilize the shortening component in a desired position by attaching the shortening component to an anchor component comprising a screw post, a bar, and/or a suture anchor implanted into a proximal tibia near the meniscus.

In further embodiments, the shortening component may be configured to be inserted between a bisected meniscal root such that the curved channel on the exterior surface of the shortening component accommodates a passage of a portion of the bisected meniscal root. The fixation component may be configured to pass through a center of the shortening component to affix the shortening component in place between the bisected meniscal root. The shortening component may include two or more support members configured to support an exterior portion of the bisected meniscal root. The shortening component may include three or more prongs extending laterally from the shortening component such that at least one of the prongs is movable to enable the path of the at least one portion of the meniscus to be altered. The shortening component may be composed from one of: stainless steel, nickel titanium alloy, titanium, cobalt-chrome, a polymer, a ceramic material, and a composite material.

According to some examples, a method to reposition a meniscus to achieve partial unloading of an ipsilateral tibiofemoral articulation may be provided. An example method may include inserting a shortening component adjacent to a meniscal root, the shortening component configured to alter a path of at least one portion of the meniscus in order to reduce a functional length of the meniscal root such that at least a partial unloading of the ipsilateral tibiofemoral articulation is achieved. The shortening component may include a semi-circular channel on an interior surface or a curved channel on an exterior surface of the shortening component configured to accommodate a passage of the meniscal root. The example method may also include affixing the shortening component to a portion of a tibia adjacent to the meniscal root employing a fixation component.

In other examples, an angle of the semi-circular channel or the curved channel of the shortening component may be adjusted to adjust the functional length of the meniscal root, and the shortening component may be rotated to adjust the functional length of the meniscal root. Two or more shortening components may be positioned such that paths of at least two or more portions of the meniscus are altered to a substantially same degree in order to reduce a strain across the meniscus. The shortening component may be positioned near one of: an anterior meniscal root of a medial meniscus, a posterior meniscal root of a medial meniscus, an anterior meniscal root of a lateral meniscus, or a posterior meniscal root of a medial meniscus.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various compositions, methods, systems, and devices are described in terms of “comprising” various components or steps (interpreted as meaning “including, but not limited to”), the compositions, methods, systems, and devices can also “consist essentially of” or “consist of” the various components and steps, and such terminology should be interpreted as defining essentially closed-member groups.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A meniscal repositioning device to achieve unloading of an ipsilateral tibiofemoral articulation, the meniscal repositioning device comprising:
a shortening component comprising a semi-circular channel on an interior surface of the shortening component, the semi-circular channel configured to accommodate a passage of a meniscal root, wherein the shortening component is configured to alter a path of at least one portion of a meniscus in order to reduce a functional length of the meniscal root such that at least a partial unloading of the ipsilateral tibiofemoral articulation is achieved; and
a fixation component configured to affix the shortening component to one or more of: a portion of a tibia adjacent to the meniscal root, an anterior portion of the meniscal root, and a posterior portion of the meniscal root.

2. The meniscal repositioning device of claim 1, wherein the at least one portion of the meniscus is drawn inward towards a midline of a knee to induce a distraction force in one of a medial compartment and a lateral compartment of the knee between a medial meniscus and a lateral meniscus.

3. The meniscal repositioning device of claim 1, wherein the semi-circular channel includes a series of smooth contours to facilitate path alteration of the at least one portion of the meniscus as the meniscal root passes through the semi-circular channel.

4. The meniscal repositioning device of claim 1, wherein the fixation component includes an anchor component attached to an inferior surface of the semi-circular channel, the anchor component configured to enable fixation of the shortening component to the tibia through a bone screw inserted through a corresponding hole in the anchor component.

5. The meniscal repositioning device of claim 4, wherein an inferior surface of the anchor component includes a series of prongs to facilitate fixation to the adjacent tibia.

6. The meniscal repositioning device of claim 4, wherein an inferior surface of the anchor component includes an osteoconductive surface to promote bone growth to facilitate fixation with the adjacent tibia.

7. The meniscal repositioning device of claim 1, wherein the shortening component includes at least two semi-circular channels and a connecting element between the at least two semi-circular channels to enable bridging of one of: the anterior portion of the meniscal root of a medial meniscus and the anterior portion of the meniscal root of a lateral meniscus, and the posterior portion of the meniscal root of the medial meniscus and the posterior portion of the meniscal root of the lateral meniscus.

8. The meniscal repositioning device of claim 7, wherein the shortening component is one of:
applied to the anterior portion of the meniscal root of the medial meniscus such that the shortening component is affixed to the anterior portion of the meniscal root of the lateral meniscus through the fixation component; and
applied to the posterior portion of the meniscal root of the medial meniscus such that the shortening component is affixed to the posterior portion of the meniscal root of the lateral meniscus through the fixation component.

9. The meniscal repositioning device of claim 1, wherein the shortening component includes an adjustable loop of suture formed around or between one of: the anterior portion of the meniscal root of a medial meniscus and the anterior portion of the meniscal root of a lateral meniscus, and the posterior portion of the meniscal root of the medial meniscus and the posterior portion of the meniscal root of the lateral meniscus.

10. The meniscal repositioning device of claim 1, wherein the shortening component and fixation component are integrated into a single structure.

11. A meniscal repositioning device to achieve unloading of an ipsilateral tibiofemoral articulation, the meniscal repositioning device comprising:
a shortening component comprising a curved channel on an exterior surface of the shortening component, the curved channel configured to accommodate a passage of a meniscal root, wherein the shortening component is configured to alter a path of at least one portion of a meniscus in order to reduce a functional length of a meniscal root such that at least a partial unloading of the ipsilateral tibiofemoral articulation is achieved; and
a fixation component configured to affix the shortening component to a portion of a tibia adjacent to the meniscal root.

12. The meniscal repositioning device of claim 11, wherein the shortening component includes a mating female Morse taper to facilitate fixation of the shortening component to the adjacent tibia through the fixation component.

13. The meniscal repositioning device of claim 12, wherein the fixation component is a threaded bone screw to facilitate anchorage in the adjacent tibia, and includes a male Morse taper at a proximal end.

14. The meniscal repositioning device of claim 13, wherein the male Morse taper of the fixation component is configured to permit relative rotation of the shortening component to obtain a desired path alteration of the at least one portion of the meniscus.

15. The meniscal repositioning device of claim 13, wherein the male Morse taper of the fixation component is further configured to permit a locking mechanism to fix the shortening component in a particular position.

16. The meniscal repositioning device of claim 11, wherein the shortening component includes two or more recesses for mating with a driver to enable mechanical rotation of the shortening component.

17. The meniscal repositioning device of claim 11, wherein the fixation component includes a series of sutures accommodated by a plurality of recesses in the shortening component configured to stabilize the shortening component in a desired position by attaching the shortening component to an anchor component comprising one of: a screw post, a bar, or a suture anchor implanted into a proximal tibia near the meniscus.

18. The meniscal repositioning device of claim 11, wherein the shortening component is configured to be inserted between a bisected meniscal root such that the curved channel on the exterior surface of the shortening component accommodates a passage of a portion of the bisected meniscal root.

19. The meniscal repositioning device of claim 18, wherein the fixation component is configured to pass through a center of the shortening component to affix the shortening component in place between the bisected meniscal root.

20. The meniscal repositioning device of claim 18, wherein the shortening component includes two or more support members configured to support an exterior portion of the bisected meniscal root.

21. The meniscal repositioning device of claim 11, wherein the shortening component includes three or more prongs extending laterally from the shortening component such that at least one of the prongs is movable to enable the path of the at least one portion of the meniscus to be altered.

22. A method to reposition a meniscus to achieve partial unloading of an ipsilateral tibiofemoral articulation, the method comprising:
inserting a shortening component adjacent to a meniscal root, the shortening component configured to alter a path of at least one portion of the meniscus in order to reduce a functional length of the meniscal root such that at least a partial unloading of the ipsilateral tibiofemoral articulation is achieved, wherein the shortening component includes one of a semi-circular channel on an interior surface and a curved channel on an exterior surface of the shortening component configured to accommodate a passage of the meniscal root; and
affixing the shortening component to a portion of a tibia adjacent to the meniscal root employing a fixation component.

23. The method of claim 22, further comprising one or more of:
altering an angle of one of the semi-circular channel and the curved channel of the shortening component to adjust the functional length of the meniscal root; and
rotating the shortening component to adjust the functional length of the meniscal root.

24. The method of claim 22, further comprising:
positioning two or more shortening components such that paths of at least two or more portions of the meniscus are altered to a substantially same degree in order to reduce a strain across the meniscus.

25. The method of claim 22, further comprising:
positioning the shortening component near one of: an anterior meniscal root of a medial meniscus, a posterior meniscal root of a medial meniscus, an anterior meniscal root of a lateral meniscus, or a posterior meniscal root of a medial meniscus.
